(19) United States
(12) Patent Application Publication
(10) Pub. No.: US 2025/0391565 A1
(43) Pub. Date: Dec. 25, 2025

(54) SYSTEM AND METHOD FOR TRAINING A MACHINE LEARNING MODEL TO SCREEN FOR A MEDICAL CONDITION BY PRE-PROCESSING TRAINING DATA TO REMOVE INDICIA OF THE HEALTH CONDITION

(71) Applicant: Santosh Yogendra SHAH, Mumbai (IN)

(72) Inventor: Santosh Yogendra SHAH, Mumbai (IN)

(21) Appl. No.: 19/241,315

(22) Filed: Jun. 17, 2025

Related U.S. Application Data
(60) Provisional application No. 63/661,836, filed on Jun. 19, 2024.

(51) Int. Cl. G16H 50/20; G06N 20/00; G16H 10/20; G16H 10/60; G16H 30/40; G16H 50/70

(52) U.S. Cl. CPC G16H 50/20; G06N 20/00; G16H 10/20; G16H 10/60; G16H 30/40; G16H 50/70

(57) ABSTRACT

A processor-implemented method for training a machine learning model to screen for a health condition may include obtaining medical data from a population of patients, pre-processing the medical data to remove indicia of a health condition, labeling encounters of the medical data according to whether the health condition is present, and training a machine learning model on the pre-processed, labeled medical data to screen for the health condition.

### CROSS-REFERENCE TO RELATED APPLICATIONS

This application claims the benefit of U.S. Provisional Application No. 63/661,836, filed Jun. 19, 2024, the contents of which are incorporated herein by reference in their entirety.

### BACKGROUND

In the realm of healthcare diagnostics, cancer screening plays a crucial role in early detection and prevention of various types of malignancies. Despite significant advancements, the field of cancer screening remains an area of active research due to the potential for earlier and more accurate identification of cancerous cells.

Currently, cancer screening relies heavily on invasive procedures such as colonoscopies alongside less intrusive methods like blood tests to identify specific biomarkers indicative of cancer, mammograms and Positron Emission Tomography (PET) scans. These techniques, though effective, come with their own limitations. For instance, PET scans require a high level of radiopharmaceutical tracers and can be expensive, limiting widespread use. Colonoscopies, although effective, can be uncomfortable for patients due to the procedure's invasive nature. Mammograms, essential for breast cancer screening, have known risks associated with radiation exposure. Furthermore, these tests are typically performed based on age and risk factors, which means some individuals may not get screened until it is too late.

Late diagnosis often results in reduced treatment options and poorer outcomes for patients. Cancers diagnosed in the later stages lead to higher mortality rates compared to those detected earlier. There is an urgent need for alternative, non-invasive, and cost-effective approaches to cancer screening.

Moreover, researchers and clinicians alike acknowledge the necessity for frequent, accessible, and personalized screening to ensure better health outcomes for individuals. Nonetheless, current practices cannot meet these demands effectively. There is a critical gap between the demand for cancer screening and the availability of resources.

Given these challenges, there exists a pressing need to develop innovative solutions that overcome the shortcomings of existing methods. There is a need to create a more accessible, less invasive, and proactive approach to cancer screening that catches diseases earlier and saves lives. A wealth of research has explored various strategies to enhance cancer screening capabilities, including machine learning algorithms, computer vision techniques, and AI applications. However, none of these innovations have fully addressed the issue of accessibility, cost, and frequency.

### SUMMARY

A processor-implemented method for training a machine learning model to screen for a health condition may include obtaining medical data from a population of patients, wherein the medical data comprises text data, audio data, and image data, and wherein the text data, audio data, and image data are associated with encounters of the patients; pre-processing the medical data, comprising: removing indicia of a health condition from the text data; converting the audio data to text, and removing the indicia of the health condition from the text; and extracting features from the image data, labeling the encounters according to whether the health condition is present by extracting information regarding whether the health condition exists from the medical data, and inferring whether the health condition is present at each of the encounters based on the extracted information; and training a machine learning model on the pre-processed, labeled medical data to screen for the health condition.

The machine learning model may be used to screen a patient for the health condition based on medical data of the patient. The method may further include updating the machine learning model by training the machine learning model on additional pre-processed, labeled medical data.

A processor-implemented method for training a machine learning model to screen for a health condition may include obtaining medical data from a population of patients; pre-processing the medical data to remove indicia of a health condition; labeling encounters of the medical data according to whether the health condition is present; and training a machine learning model on the pre-processed, labeled medical data to screen for the health condition.

The health condition may include cancer. The medical data may include summaries of statuses of the patients, audio data of the patients, image data of the patients, or video data of the patients. The indicia of the health condition may include diagnoses of the health condition and information that indicates that the health condition is present. The information that indicates that the health condition is present may include a medical procedure necessitated by the health condition or a symptom of the medical condition. The pre-processing of the medical data may include scrubbing the indicia of the health condition from the medical data using a rule-based algorithm. The labeling of the encounters of the medical data may include extracting information regarding whether the health condition exists from the medical data, and inferring whether the health condition is present at each of the encounters based on the extracted information. Each of the encounters may be associated with a respective health check of a respective patient of the patients. The method may further include in response to a most recent encounter of the respective patient being labeled as positive for the health condition, labeling encounters of the respective patient within a time period of the most recent encounter as positive for the health condition, and excluding all encounters of the respective patient outside of the time period from the training. further comprising, in response to an encounter of the respective patient being labeled as positive for the health condition, excluding all subsequent encounters of the respective patient from the training. further comprising, in response to an encounter of the respective patient being labeled as negative for the health condition, labeling all prior encounters of the respective patient as negative for the health condition.

A system for training a machine learning model to screen for a health condition may include one or more processors configured to: obtain medical data from a population of patients; pre-process the medical data to remove indicia of a health condition; label encounters of the medical data according to whether the health condition is present; and train a machine learning model on the pre-processed, labeled medical data to screen for the health condition.

The health condition may include cancer. The medical data may include summaries of statuses of the patients, audio data of the patients, image data of the patients, or video data of the patients. The indicia of the health condition may include diagnoses of the health condition and information that indicates that the health condition is present. The information that indicates that the health condition is present may include a medical procedure necessitated by the health condition or a symptom of the medical condition. The one or more processors may be further configured to pre-process the medical data by scrubbing the indicia of the health condition from the medical data using a rule-based algorithm. The one or more processors may be further configured to label the encounters of the medical data by extracting information regarding whether the health condition exists from the medical data, and inferring whether the health condition is present at each of the encounters based on the extracted information. Each of the encounters may be associated with a respective health check of a respective patient of the patients. The one or more processors may be further configured to, in response to a most recent encounter of the respective patient being labeled as positive for the health condition, label encounters of the respective patient within a time period of the most recent encounter as positive for the health condition, and exclude all encounters of the respective patient outside of the time period from the training. The one or more processors may be further configured to, in response to an encounter of the respective patient being labeled as positive for the health condition, exclude all subsequent encounters of the respective patient from the training. The one or more processors may be further configured to, in response to an encounter of the respective patient being labeled as negative for the health condition, label all prior encounters of the respective patient as negative for the health condition.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 is a schematic diagram of a system for screening for a health condition, according to an embodiment of the present disclosure;

FIG. 2 is a schematic illustration of encounters associated with patients, according to an embodiment;

FIG. 3 is a schematic illustration of a process of labeling encounters, according to an embodiment;

FIG. 4 is a schematic illustration of medical data of an encounter, according to an embodiment;

FIG. 5 is a schematic illustration of a process of converting and scrubbing medical data of an encounter;

FIG. 6 is a schematic illustration of a process of uploading the labeled encounters, according to an embodiment; and

FIG. 7 is a flow diagram of a method of training a machine learning model to screen for a health condition, according to an embodiment.

### DETAILED DESCRIPTION

It should be understood at the outset that although illustrative implementations of one or more embodiments are illustrated below, the disclosed systems and methods may be implemented using any number of techniques, whether currently known or not yet in existence. The description that follows includes example systems, methods, techniques, and program flows that embody aspects of the disclosure. However, it is understood that this disclosure may be practiced without these specific details. For brevity, well-known steps, protocols, structures, and techniques have not been shown in detail in order not to obfuscate the description. The disclosure should in no way be limited to the illustrative implementations, drawings, and techniques illustrated below, but may be modified within the scope of the appended claims along with their full scope of equivalents.

The system and method of the present disclosure may include applying artificial intelligence (AI) and machine learning (ML) techniques to analyze and interpret large volumes of routine medical encounter data. This data may include patient's clinical notes from their encounters, routine blood tests, X-ray images, CT scans, MRI scans, ultrasound examinations, mammograms, or other medical data. By leveraging advanced computational capabilities, detection of conditions such as cancers may be improved without specialized tests like scans, cancer blood biomarkers etc., thereby facilitating inexpensive and early detection of diseases, which may result in timely interventions and improved health outcomes for patients suffering from cancer, rare diseases, and chronic conditions such as autoimmune diseases. Particularly, the system and method may produce a probability of the disease, the mapping of that probability to whether the patient is likely to have the disease and an interpretation relating to the percentage of patients with a similar/same probability and score having the condition versus the percentage of patients with a similar/same probability and score not having the condition. In some embodiments, these percentages may be derived from the patient cohort used for training the AI/ML model and not the general population.

A system and method for early detection of cancer and chronic conditions, including autoimmune diseases, using routinely available data during medical encounters, is disclosed. The system and method may utilize AI and ML technologies to analyze various types of medical data, such as clinical notes, laboratory test results, imaging studies, and signal data such as EKG data. By processing and analyzing this diverse dataset, the system can identify patterns and trends that may indicate the presence of early-stage cancer or chronic conditions. As a result, the system has the potential to provide earlier diagnoses, leading to better prognosis and management of these conditions.

In some embodiments, the system includes AI-assisted cancer screening software capable of analyzing medical data from multiple sources to detect cancerous trends and anomalies at an early stage, offering personalized screening recommendations based on individual risk profiles. By employing machine learning algorithms, this system may enable non-invasive, frequent, and cost-effective cancer screening, potentially improving overall patient care and outcomes.

In some embodiments, the system includes a suite of software applications designed to facilitate the collection, storage, preprocessing, feature extraction, and prediction generation processes. A machine learning algorithm capable of processing vast amounts of heterogeneous data may be implemented to generate accurate and reliable predictions concerning the likelihood of an individual developing cancer or experiencing adverse health effects associated with chronic conditions. Additionally, the system may include an API based integration with the EHRs (electronic health records), and a user-friendly web-based graphical user interface (GUI) designed to enable healthcare providers and patients alike to interact with the system in real-time and view personalized reports detailing their current state of health.

The cancer screening system may include a quality control mechanism configured to regularly review and validate the accuracy and reliability of the predictions generated by the system. This may ensure that the system remains up-to-date and continues to deliver consistent and high-quality results over time. Furthermore, the system may be configured to be fully scalable and extensible, allowing for easy integration with existing electronic health records systems and other related technologies.

The AI-assisted cancer screening system may enable early detection and prevention of various types of cancers. The system may utilize deep learning models trained on large datasets of EHR data, enabling it to identify patterns and correlations that may not be immediately apparent to human clinicians. These patterns can include laboratory test results, clinical notes, demographic data, and other relevant data points. By analyzing this data, the system can provide physicians with actionable insights regarding potential risks and recommended follow-up procedures, allowing for earlier intervention and improved patient outcomes.

For example, the system may be implemented in population-scale screening programs. These programs can identify cancers at an early stage, yet they may require extensive resources and manpower to analyze the massive amounts of data generated. The system can significantly reduce the workload on healthcare professionals by automatically analyzing EHR data and flagging individuals who may be at higher risk for certain types of cancers. This not only saves time and resources but also enables healthcare providers to focus their efforts on those patients who truly need further investigation and treatment.

Furthermore, the system can be integrated into existing electronic health record systems, making it easy for healthcare providers to adopt and implement. The system can also be customized to specific populations or healthcare settings, allowing for tailored risk assessments and targeted interventions. For example, the system can consider factors such as age, gender, lifestyle choices, and genetic predispositions to provide more accurate risk predictions and personalized recommendations.

The system and method of the present disclosure may utilize data collected during standard medical interactions to identify and screen for various medical conditions, for example, cancer, autoimmune diseases, and genetic disorders. Typically, specialized tests are required to screen for these conditions. Screening tests are typically those that can be applied at scale across populations, for example, mammograms. In contrast, tests like PET scans are expensive and not as easily accessible and are therefore usually not considered screening tests. Cancer screening may depend on the site of the cancer within the body. For example, tests such as MRIs and diagnostic blood markers may be needed. Autoimmune diseases, such as rheumatoid arthritis, require tests such as the Rheumatoid Factor, while Systemic Lupus Erythematosus (SLE) necessitates ANA antibody testing. Genetic disorders often involve testing mutations in specific genes. Due to the high prevalence of these diseases compared to testing rates, many cases go undetected or are diagnosed too late. Early diagnosis significantly increases survival rates and reduces costs for healthcare payers and governments. Currently, guidelines exist for cancer testing and screening, typically based on age and gender. However, despite these guidelines, many early cases remain undetected until it is too late, leading to increased costs and potential loss of life.

The system may comprise a memory and a processor. The processor may store instructions that when executed causes the processor to leverage data from each medical interaction between a patient and the healthcare system. The data may encompass various formats, including textual data (clinical reports), routine tests (blood work, imaging studies), and multimedia recordings (EKGs, videos). The system and method may incorporate an AI model that processes this diverse data in various combinations (e.g., clinical reports alone, clinical reports and blood work, etc.). Preprocessing techniques customized to each type of data may ensure optimal feature extraction for machine learning algorithms.

Supervised machine learning may be employed, utilizing retrospective data where the labels denote the presence or absence of specific medical conditions. During the preprocessing stage, the processor may remove any instances of disease, for example cancer-related terms, from the clinical reports of patients previously diagnosed with the disease/condition. For example, terms such as ‘cancer,’ ‘carcinoma,’ and ‘malignancy’ may be removed. By doing so, the AI algorithm may be taught to identify subtle differences between patients with and without the disease/condition (e.g., cancer) using alternative features, such as combinations of symptoms, signs, findings from X-ray and other investigations that are non-specific for cancer, and/or blood test results. This may be advantageous because these distinctions might not be readily apparent to healthcare professionals. The AI model may accurately predict undetected cancer without definitive features of the cancer that would have alerted a clinician. For example, the model may be trained and validated on 196,000 patient records. For example, the AI model may have a recall >0.80 for both patients with and without the medical conditions. By improving the odds of early cancer detection, the system may offer significant advantages, such as earlier interventions, treatments, decreased mortality and morbidity, and reduced healthcare costs.

The system and method may apply AI/ML to routine medical encounter data to screen for cancer and chronic conditions. The system and method can analyze various types of medical data to identify potential signs or indicators of these conditions. By doing so, it can help healthcare professionals make more informed decisions and potentially improve patient outcomes. The system and method may advantageously provide an automated screening tool for cancer and chronic conditions using routine medical encounter data. To screen for cancer and chronic conditions, this information may be analyzed using AI and ML techniques. These techniques can help to identify patterns and relationships in the data that may indicate the presence of these conditions.

The system and method may make predictions or probability estimates for the likelihood of certain conditions such as cancer or autoimmune diseases based on the patient's medical history and other factors. Visualizations or summaries of the data that highlight important patterns or relationships may be generated. Recommendations for further testing or treatment options may be output based on the predictions and visualizations.

The data may include various types of medical records such as clinical notes, blood test results, X-ray images, ultrasound images, and mammograms. This data may be used to screen for cancer and chronic conditions, including autoimmune diseases. This data can be categorized as structured tabular data and unstructured image, text, audio, video, signal (EKG etc.) data. To perform these tasks, the AI/ML models may process the data, extract relevant information, and make predictions or decisions. Techniques may involve image processing, natural language processing, statistical modeling, and/or other techniques.

Preprocessing ensures that the data is clean, consistent, and in the correct format for the ML algorithms. Firstly, preprocessing may be applied to both continuous and categorical data. Data cleaning may be performed to handle missing values by either removing instances with missing data or filling them using methods such as mean or median imputation. Outliers may be identified and handled using various techniques such as Z-score, IQR (Interquartile Range), or Winsorizing. Normalization or scaling techniques like MinMaxScaler or StandardScaler may be applied to ensure that features have similar ranges and distributions, which can help improve model performance.

Normalization may be used when dealing with large differences in scales between different features. This technique may transform each feature so it has zero means and unit variance, ensuring equal importance in the model. Another technique that may be implemented is Feature Extraction, which may involve creating new features from existing ones, for example, calculating ratios or polynomial expansions.

Categorical data may be encoded. An encoding method that may be used is Label Encoding, in which each category is replaced by a unique integer value. However, this might lead to loss of information, especially if there is any inherent ordering within the categories. To maintain information about the hierarchy, techniques such as One-Hot Encoding may be used, where a binary column is created for each category level.

Medical images can be important for diagnosing various health conditions. However, these images are often in an unstructured format, making them difficult for machines to interpret directly. Preprocessing may prepare these images for analysis by ML models.

The first step for processing medical images may involve acquiring raw data from imaging devices such as MRI scanners, CT scanners, or X-ray machines. This data may contain noise, inconsistencies, and artifacts due to various factors such as patient motion during the scan, imperfect hardware, or environmental influences. To address these issues, initial processing techniques such as filtering, normalization, and denoising may be applied to enhance image quality. These methods may remove unwanted signals while preserving useful information, improving overall contrast and reducing artifacts that could negatively impact model performance. This may improve the accuracy of the output of the trained ML model by preventing overfitting, ensuring all feature contribute equally during optimization, and preventing the model from learning irrelevant patterns caused by noise. Additional efficiencies may also be gained in terms of storage and processing of medical images.

Segmentation refers to the process of identifying specific regions within an image based on their distinct characteristics. In medical images, this segmentation may be used to isolate structures of interest, such as tumors, lesions, or organs. Segmented regions may then be labeled, assigning each region a unique identifier that helps ML algorithms distinguish between different tissues, anomalies, or features. Accurate segmentation lays the foundation for subsequent analyses, including feature extraction and classification.

Once segmented regions have been identified and labeled, features may be extracted from the images using various computational techniques. The features may capture relevant information about the shape, texture, intensity, size, or location of the segments. For instance, texture features may describe the spatial arrangement and statistical properties of pixels within a region, while shape descriptors may quantify the geometric properties of the segmented object. These features may act as input data for ML algorithms, allowing them to learn patterns and make predictions based on the provided information.

Medical image preprocessing may include data augmentation, such as when dealing with limited datasets. Data augmentation may involve generating new synthetic samples by applying transformations, such as rotation, scaling, flipping, or cropping, to existing images. Data augmentation may increase the size and diversity of the dataset, helping to prevent overfitting, improve generalization capabilities, and enhance model robustness. By incorporating these synthetic samples into the training set, ML algorithms can gain a more comprehensive understanding of the underlying data distribution, leading to better model performance and increased accuracy.

The unstructured medical text data may be pre-processed to prepare it for ML models. In this context, unstructured medical text refers to free-form text found in various sources such as clinical notes, discharge summaries, radiology reports, and pathology reports.

The first step in preprocessing unstructured medical text data may involve cleaning and normalizing the raw text. This may include removing irrelevant information like identifiers, stop words, and punctuations. Normalization may involve converting all text to lowercase or stemming words to their base form. For instance, “diabetes mellitus” may be converted to “diabetes.” Additionally, misspelled words may be handled to ensure accurate processing. Techniques such as spell checking, lemmatization, or using dictionaries can aid in correcting errors.

Meaningful features may be extracted from the cleaned and normalized text. Feature extraction techniques like Bag of Words (BoW), Term Frequency-Inverse Document Frequency (TF-IDF), Named Entity Recognition (NER), Part-of-Speech (POS) tagging, and/or Dependency Parsing may be used. These techniques may convert text into numerical representations, which ML algorithms can understand. For example, BoW may create a matrix where each row represents a document and each column corresponds to a unique term in the corpus. Each cell may contain the frequency count of the corresponding term in the document.

In the pre-processing of the training data, cancer references may be scrubbed, for example, by name entity recognition (NER). NER may help find and identify relevant words or phrases in a piece of text. NER may automatically spot and label relevant terms such as diseases (e.g., breast cancer, diabetes); medications (e.g., paracetamol, chemotherapy); tests (e.g., pet scan, biopsy); and/or people or doctors' names, dates, and locations. For example, NER may be applied to the following sentence: “the patient underwent a pet scan, which showed lung cancer and lymph node metastasis.” NER may identify and label: pet scan (a test/procedure), lung cancer (a disease), lymph node metastasis (disease/finding). Clinical NER models may detect mentions of diseases (e.g., “adenocarcinoma”, “malignant melanoma”), procedures (e.g., “core needle biopsy”, “pet scan”), and/or findings (e.g., “atypical squamous cells”). Those diseases, procedures and findings terms that were indicative of cancer may be filtered and replaced with the empty string “” to scrub all mentions of cancer from the clinical summaries. All the remaining non-cancer terms may be kept.

Cancer indicative disease terms may include generic cancer terms (which may always be flagged) such as cancer, malignancy, malignant neoplasm, carcinoma, neoplasm, tumor/tumour, oncology, oncologic disease, cancers of unknown primary (cup), invasive cancer, and/or metastasis/metastatic disease. Organ-specific cancers (e.g., any disease that refers to cancer in a specific organ/system) may also be flagged and may include oral cancer, oropharyngeal carcinoma, laryngeal cancer, nasopharyngeal carcinoma, thyroid cancer, parotid gland tumor, lung cancer, non-small cell lung carcinoma (NSCLC), small cell lung carcinoma (SCLC), breast cancer, invasive ductal carcinoma (IDC), invasive lobular carcinoma (ILC), ductal carcinoma in situ (DCIS), triple-negative breast cancer (TNBC), HER2-positive breast cancer, esophageal carcinoma, gastric cancer, colorectal cancer (CRC), colon cancer, rectal cancer, pancreatic cancer, hepatocellular carcinoma (HCC), gallbladder cancer, cholangiocarcinoma (bile duct cancer), bladder cancer, kidney (renal cell) carcinoma, urothelial carcinoma, prostate cancer, testicular cancer, penile cancer, cervical cancer, endometrial carcinoma, uterine cancer, ovarian cancer, vulvar cancer, vaginal cancer, gestational trophoblastic disease, melanoma, basal cell carcinoma (BCC), squamous cell carcinoma (SCC), merkel cell carcinoma, leukemia (ALL, AML, CLL, CML), lymphoma (hodgkin's and non-hodgkin's), multiple myeloma, myelodysplastic syndrome (MDS), myeloproliferative neoplasms, glioblastoma multiforme (GBM), astrocytoma, meningioma (malignant), medulloblastoma, ependymoma, CNS lymphoma, mesothelioma, sarcoma (osteosarcoma, chondrosarcoma, leiomyosarcoma, neuroendocrine tumor (net), carcinoid tumor, and/or germ cell tumor.

Precancerous/high-risk conditions may also be flagged and may include dysplasia (high-grade/low-grade), atypical hyperplasia, carcinoma in situ (e.g., CIS bladder, CIN III), barrett's esophagus with dysplasia, monoclonal gammopathy of undetermined significance (MGUS), smoldering multiple myeloma, lichen sclerosus (in context of vulvar cancer risk). Staging/biomarker terms may also be flagged (NER tags that may not be diseases but imply cancer) and may include TNM staging (e.g., T2N1MO), stage I-IV, gleason score (for prostate), HER2, ER/PR (breast), CA-125, CEA, PSA, AFP, etc., FDG-avid lesion, SUV max (from pet scan). Treatment-associated NER markers (e.g., terms that suggest ongoing or past cancer treatment) may also be flagged and may include chemotherapy, radiotherapy/radiation, immunotherapy, oncologic surgery, mastectomy/lumpectomy/prostatectomy, bone marrow transplant, and/or targeted therapy (e.g., EGFR, ALK inhibitors).

Cancer-indicative procedures may also be flagged via NER. Diagnostic procedures (e.g., strong indicators of suspicion or confirmation of cancer) may be flagged and may include biopsy (general term), core needle biopsy, fine needle aspiration (FNA/FNAC), excisional biopsy, incisional biopsy, punch biopsy, shave biopsy, bone marrow biopsy, endoscopic biopsy (e.g., gastric, bronchial), image-guided biopsy (ct-guided, ultrasound-guided), tru-cut biopsy, stereotactic biopsy, pap smear (if abnormal), cytopathology, pleural fluid cytology, ascitic fluid cytology, urine cytology, CSF cytology, nipple aspirate cytology, bronchial washings/brushings, histopathology, frozen section analysis, immunohistochemistry (IHC), molecular pathology, fluorescence in situ hybridization (fish), next-gen sequencing (NGS), liquid biopsy, and/or pathology review.

Cancer staging/imaging procedures (e.g., used to assess disease extent, staging, or recurrence) may also be flagged and may include pet scan/PET-CT, bone scan, MIBG scan, SPECT, gallium scan, FDG PET (fluorodeoxyglucose), mammography/digital mammogram, colonoscopy/sigmoidoscopy, cystoscopy, bronchoscopy, laryngoscopy, and/or hysteroscopy. Molecular/genetic/biomarker testing terms (e.g., highly suggestive of cancer when ordered with diagnostic intent) may also be flagged and may include BRCA1/BRCA2 testing, KRAS/NRAS/EGFR mutation testing, ALK, BRAF, HER2/NEU, MSI/MMR status (microsatellite instability), liquid biopsy (circulating tumor DNA), tumor mutation burden (TMB), PD-L1 testing, and/or oncotype DX/mammaprint.

Cancer treatment procedures terms (e.g., indicating confirmed diagnosis or treatment phase) may also be flagged and may include, tumor resection, lumpectomy, mastectomy (total, partial), prostatectomy, hysterectomy, colectomy, nephrectomy, thyroidectomy, debulking surgery, sentinel lymph node biopsy/axillary dissection, mediastinoscopy, craniotomy for tumor, external beam radiation, IMRT (intensity-modulated radiation therapy), stereotactic radiosurgery (SRS/cyberknife/gamma knife), brachytherapy, whole brain radiation therapy (WBRT), chemotherapy infusion, intrathecal chemotherapy, oral chemotherapy, neoadjuvant/adjuvant chemotherapy, port catheter insertion (port-a-cath), monoclonal antibodies (e.g., pembrolizumab, trastuzumab), CAR-T therapy, EGFR/ALK/BRAF inhibitors, bone marrow transplant (BMT), hematopoietic stem cell transplant (HSCT), and/or autologous/allogeneic transplant. Structured clinical procedures/referrals (e.g., appearing in summaries, can be context indicators) may also be flagged and may include oncology referral/oncologist consult, tumor board discussion, palliative care initiation, clinical trial enrollment (for cancer), multidisciplinary team (MDT) planning, and/or cancer registry submission.

Pathology/histology findings (e.g., phrases that often indicate malignancy or suspicious tissue behavior) may also be flagged and may include atypical cells, malignant cells, carcinoma in situ, invasive carcinoma, high-grade dysplasia, low-grade dysplasia, poorly differentiated cells, moderately differentiated cells, well-differentiated tumor, undifferentiated neoplasm, neoplastic cells present, tumor cells seen, positive for malignancy, abnormal mitotic figures, increased nuclear-cytoplasmic ratio, necrotic tumor areas, hyperchromatic nuclei, nuclear pleomorphism, gland-forming lesion, papillary structures, solid nests of cells, lymphovascular invasion, perineural invasion, mucin-producing cells, signet-ring cells, sheets of small round blue cells, and/or reed-sternberg cells (hodgkin's lymphoma).

Imaging findings (radiology reports) (e.g., phrases imply masses, suspicious features, or metastatic spread) may also be flagged and may include suspicious mass, space-occupying lesion, enhancing lesion, irregular margins, spiculated mass, hypodense lesion, hyperdense lesion, heterogeneous mass, soft tissue mass, solid-cystic lesion, T2 hyperintense lesion, restricted diffusion, enhancement post-contrast, ill-defined lesion, necrotic center, calcifications (when atypical), lytic bone lesions, sclerotic bone lesions, multiple nodules, lung metastasis, liver metastases, peritoneal nodules, pleural thickening/effusion (suspicious), brain metastasis, FDG-AVID lesion (from PET scan), and/or SUV max >x (e.g., >2.5). Structured clinical impressions (e.g., detected via NER/REGEX in clinical summaries) may also be flagged and may include impression: suspicious for malignancy; assessment: probable carcinoma; diagnosis: suggestive of cancer; plan: refer to oncology, final diagnosis: adenocarcinoma; and/or working diagnosis: malignancy.

The data may be encoded and transformed into a suitable format for feeding it into ML models. Encoding can include one-hot encoding, binary encoding, or label encoding based on the requirements. Transformation techniques such as Principal Component Analysis (PCA), Singular Value Decomposition (SVD), or T-Distributed Stochastic Neighbor Embedding (t-SNE) can also be applied to reduce dimensionality while retaining important patterns. Data augmentation techniques such as oversampling, undersampling, and/or synthetic data generation can be employed to increase the size of the dataset and address class imbalance issues.

Unstructured medical text data may be pre-processed for the ML model. When dealing with sensitive medical information, it is important to ensure privacy while maintaining accuracy. Text data may be prepared from various sources such as medical case reports, blood test reports, and imaging reports. Raw data may be imported and cleaned by removing irrelevant tags, stop words, and/or punctuation marks. The text may be normalized by converting it to lowercase or lemmatized. This step can make standardization and further processing more efficient. Once cleaned, techniques such as tokenization and stemming may be employed to break down complex terms into simpler components.

Instances of cancer-related terminology in the positive (e.g., cancer-present) class may be replaced with neutral terms. Synonyms such as malignancy, neoplasm, tumor, carcinoma, and oncology may be targeted for replacement with non-specific terms to maintain the challenge for the model. A list of these terms may be created beforehand to efficiently search and replace them throughout the text. In some embodiments, no text data from the negative (e.g., no cancer) class will undergo any modifications. This may balance classes for equal representation in the training dataset, and allow the model to learn the distinction between normal and abnormal conditions effectively based on the original text.

After completing the preprocessing steps, advanced natural language processing methods may be applied, such as named entity recognition and/or part-of-speech tagging, to extract meaningful features from the text data. These extracted features may serve as inputs for ML algorithms to learn and make accurate predictions. Additionally, other data types may be incorporated such as numerical lab results from blood tests or image analysis features for comprehensive modeling.

Unstructured medical audio data may be preprocessed for analysis using ML algorithms. Medical audio data, such as patient interviews, consultations, or diagnostic tests, come in various formats and qualities, which can make them difficult to work with directly. Preprocessing may involve cleaning, normalizing, and transforming raw audio data into a format that can feed into ML models effectively. In some embodiments, speech-to-text conversion may be used. This technique may extract text from spoken language by applying various signal processing techniques such as noise reduction, spectral subband energy, and pattern recognition algorithms. Cleaning the transcribed text may include removing background noise, irrelevant information, and inconsistent terminology, which can ensure high-quality data for downstream tasks. This may reduce the volume of unnecessary or redundant data, learning to faster parsing and lower memory usage. It may also improve the efficiency of model training.

Feature extraction may be used to convert raw data into numerical representations suitable for ML models. Feature extraction methods such as Mel Frequency Cepstral Coefficients (MFCCs), Linear Predictive Coding (LPC), and/or Perceptual Linear Precision (PLP) may be used. These features may capture important aspects of the audio signal, allowing ML models to learn meaningful patterns and make accurate predictions.

Augmentation techniques can be used to enhance the quality and size of preprocessed medical audio data, increasing the robustness and generalization capability of ML models. Data augmentation strategies such as adding artificial background noise, simulating different recording conditions, and/or altering pitch and tempo may be used. By generating additional synthetic data, these techniques can increase the variety and richness of available data, which can improve model performance and reduce overfitting issues.

Preprocessing of unstructured medical signal data, specifically Electrocardiogram (EKG) data helps to prepare the medical signal data for analysis using ML algorithms. EKG data, which represents the electrical activity of the heart, may comes in noisy, unevenly sampled, and irregular formats, which can necessitate careful handling before feeding it into ML models. The first step in preprocessing EKG data may be to filter out unwanted noise and artifacts. This may involve applying bandpass filters to remove irrelevant frequency components, notch filters to eliminate powerline interference, or wavelet denoising to remove random noise. Additionally, baselining the data can be corrected for systematic offsets, ensuring consistent measurements across all samples.

A component of EKG data preprocessing may be signal filtering. Various filters, such as Butterworth, Chebyshev Type II, and/or Infinite Impulse Response (IIR) filters can be employed to eliminate baseline wander, powerline interference, and other types of noise. Additionally, adaptive filters can learn and adaptively adjust their characteristics based on the input data, further enhancing noise removal capabilities.

The EKG signals may be segmented into individual beats or epochs. This process may allow for focus on specific parts of the data relevant for analysis, rather than dealing with the entire continuous stream. Techniques may be used for beat detection, for example, template matching, pan-tomographic QRS complex detection, and/or RR interval measurement. Proper segmentation can ensure that ML models are provided with well-defined input examples.

Feature extraction helps convert raw EKG data into numerical representations suitable for ML models. Feature extraction models that may be applied to EKG data can include time domain features (such as QRS complex duration, PR interval, RR interval, and heart rate variability), frequency domain features (e.g., spectral power in different bands), wavelet-based features, and/or time-frequency domain features (e.g., Short-Term Fourier Transform (STFT) coefficients). These extracted features can represent important characteristics of the EKG signals and enable ML models to identify distinct patterns related to various cardiac conditions.

Data augmentation techniques can also improve the quality and quantity of preprocessed EKG data, enhancing the overall performance of ML models. Augmentation strategies can include simulated physiological variability, such as adding heart rate changes, introducing baseline wander, or mimicking ectopic beats. Augmentation strategies may introduce synthetic variations such as adding Gaussian noise or altering temperature, heart rate, and/or body position during data acquisition. Augmented data can provide increased variation and diversity, helping ML models to better adapt to real-world variations and reducing the risk of overfitting.

AI algorithms can help to transform unstructured data from electronic health records (EHRs) into valuable insights that aid clinicians in diagnosis and treatment planning. Text classification models can be employed to screen large volumes of medical text, such as case reports, for specific conditions like cancer.

The system and method may use a Naive Bayes (NB) algorithm. This probabilistic method may assume that each feature (e.g., word or term) in the document is independent of others given the class label. The NB algorithm may learn patterns by calculating conditional probabilities between features (e.g., terms) and classes based on the training dataset.

The system and method may additionally or alternatively use Support Vector Machines (SVM), which may use labeled examples to find the optimal boundary that separates different classes. SVM can handle high-dimensional datasets and nonlinear relationships between features, making it suitable for complex cases. SVM may analyze the input vectors (e.g., term frequencies) of case reports and may look for the hyperplane with the largest margin that maximally separates positive (e.g., cancer) and negative (non-cancer) instances.

The system and method may additionally or alternatively use Random Forest Classifier (RFC). RFC may refer to an ensemble learning method that constructs multiple decision trees from random subsets of the dataset. Each tree may cast a vote for a particular class, and the final prediction may be determined by the majority class among all votes. RFC can offer robustness to outliers and overfitting issues. RFC may build several trees on randomly sampled feature sets and combine their outputs to classify new cases.

In some embodiments, AI algorithms can help in medical multimodal classification, specifically for screening cancer from medical case reports and structured tabular data obtained during routine medical encounters. ML models may be used for this purpose, for example, Naive Bayes, Logistic Regression, Decision Trees, Random Forest, Support Vector Machines (SVM), and/or Neural Networks. Furthermore, deep learning models such as Long Short-Term Memory (LSTM) networks or Convolutional Neural Networks (CNN) can be utilized to effectively capture complex patterns within the textual data.

When developing ML models, validating the model's performance on separate datasets helps to ensure that the ML model generalizes well to new, unseen data. The available dataset may be divided into three main parts: training set, validation set, and test set. While the training set is used to fit the model with the provided features and learn the underlying patterns, the validation set may serve two primary purposes: monitoring the model's progress during training and fine-tuning hyperparameters if necessary. The test set may be used to evaluate the final performance of the trained model.

The validation may allow for monitoring of the model's behavior throughout the training process. During each epoch, the model may be evaluated on the validation set, and the corresponding performance metrics like accuracy, precision, recall, Fl score, etc., may be recorded. This information can help determine whether the current state of the model is improving or deteriorating and guides the choice of stopping criteria. For instance, if the model's performance on the validation set starts degrading after a certain point, training may be stopped to avoid overfitting the model to the training data.

Moreover, the validation set may be used for hyperparameter tuning. Hyperparameters can significantly impact the overall performance of the ML model. Fine-tuning these values may require evaluating multiple combinations of hyperparameters and selecting the best ones based on the model's performance on the validation set. Once a suitable set of hyperparameters has been identified, they can be applied to the test dataset to assess the final performance of the trained model. The test dataset may consist of completely new, unseen data to provide an accurate assessment of how the model performs in practice.

Before feeding the data into the chosen algorithm, preprocessing may be performed to cleanse and prepare it for analysis. For text data, various cleaning tasks may be performed such as removing stop words, punctuation marks, numbers, and special characters. Stemming or lemmatization may be applied to normalize words based on their root form. Structured data may undergo similar cleansing steps, but may require additional transformation to make the structured data suitable for input into the model.

Feature extraction may be used to convert raw data into a format that machines can understand. For text data, BoW or TF-IDF vectors may be commonly used. For structured data, features could be statistical measures or derived values, such as mean, median, mode, standard deviation, or another measured or derived value. Combining both modalities can create a richer feature sets for improved classification results.

Once all features are extracted, they may be fed into the selected algorithm for training. A 10-fold cross-validation may be used to select the best model. This may involve partitioning the available data into ten equal parts, called “folds.” Then, the model may be fit nine times using nine folds for training, while the remaining fold is left aside for testing. Afterward, the process may be repeated for each fold until every fold has once served as the test set. Finally, the average performance across all ten runs may be computed.

The 10-fold cross-validation may be advantageous because of its ability to provide a more reliable estimate of the model's generalization error. Instead of relying on just one test set, which could potentially lead to biased estimates, 10-fold cross-validation may average the performance over multiple iterations. Moreover, it can also help check whether the dataset is consistent enough to train a robust model. If the variation between the performances of different folds is significant, then it suggests that there might be issues with the dataset, such as inconsistent features or insufficient data.

The 10-fold cross-validation can be applied to various machine learning tasks, including regression problems, time series forecasting, and/or binary/multi-class classification. The dataset may be divided into ten equal parts, and the cross-validation procedure may be applied. At each iteration, accuracy, precision, recall, and F1 score on the test set may be calculated to obtain an assessment of the model's performance. Once completed, the results from all the ten iterations may be combined to derive the overall evaluation metrics. This approach may provide a comprehensive understanding of how well the chosen model performs on unseen data and may contribute to selecting the best possible model for a specific problem. Although ten folds is used as an example, any number of folds is within the scope of the present disclosure.

The ML model may be trained using optimization to fine-tune the parameters to achieve optimal performance. Optimization may refer to the process of adjusting the model's parameters in such a manner that the loss function reaches its minimum. Hyperparameters may be used. Hyperparameters may be configuration variables that are selected before the training process begins, such as the learning rate, batch size, regularization strength, and number of hidden layers. These values may be manually or algorithmically determined through hyperparameter tuning.

Gradient Descent (GD) may be used. GD may be an iterative optimization algorithm where the goal is to find the values of the model parameters that minimize the loss function by updating them in small steps, referred to as learning rates. Each update step may be calculated based on the gradient of the loss function, which points in the direction of steepest descent. Through repeated updates, the model's parameters may converge towards their optimal values.

GD, such as Stochastic Gradient Descent (SGD), Mini-Batch Gradient Descent, and Adam Optimizer, may be used to improve convergence speed and prevent getting trapped in suboptimal solutions. SGD may sample random instances from the dataset at each iteration for computing gradients. Mini-batch gradient descent may compute gradients based on small batches rather than the entire dataset. An Adam Optimizer may adapt the learning rate during training by estimating both first-order and second-order moments of the gradients.

Backpropagation may also be used. The backpropagation algorithm may calculate the gradient of the loss function with respect to each weight in the network, enabling the computationally efficient implementation of multi-layer neural networks using GD. By propagating the errors backward from the output layer to the input layer, the weights may be updated in the opposite direction, thus allowing the network to learn complex representations from raw data.

Hyperparameter tuning may be used. Hyperparameter tuning may be the systematic process of finding the optimal combination of hyperparameters for a given ML model. It may maximize predictive performance while minimizing computational effort required. Common techniques for hyperparameter tuning may include Grid Search, Random Search, Bayesian Optimization, and Genetic Algorithm. Grid search may involve evaluating all possible combinations within a predefined range of hyperparameters, while random search may explore randomly sampled combinations. Bayesian optimization may use probabilistic models to guide the search toward the most promising areas of the parameter space. Genetic algorithms may employ evolutionary strategies inspired by natural selection.

The model's performance may be evaluated using appropriate evaluation metrics such as Accuracy, Precision, Recall, F1 Score, Area Under ROC Curve, or Confusion Matrix depending on the problem statement. Accuracy may be the proportion of correct predictions made by the model out of all the instances in the dataset. The metric may measure the overall performance of the classifier. Precision may be the ratio of true positive predictions (correctly identified positives) to the total predicted positives. In other words, it may measure how many of the positive predictions were actually correct. A high precision implies that most of the positive predictions were indeed actual positives. Recall (e.g., Sensitivity or True Positive Rate) may measures the proportion of actual positives that were correctly identified as such. The percentage of the real positive cases detected may be measured. A high recall implies that the majority of the real positive cases were correctly identified. F1 score may be used. FI score may be the harmonic mean of precision and recall. It may balance both of these measures. Area Under the ROC Curve (AUC-ROC) may be used. AUC-ROC may represent the entire two-dimensional area underneath the entire ROC curve. The closer the AUC value is to 1, the more effective the classifier may be at separating between positive and negative classes.

A confusion matrix may be used. The confusion matrix may be a table layout that allows visualization of prediction results on a given data testing set. It may consist of rows and columns, with row labels representing the actual outcomes and column labels representing the predicted outcomes. Each cell in the matrix may indicate the number of observations belonging to the intersection of the row label and column label. The diagonal elements may represent true positives and true negatives. Off-diagonal elements may represent false positives and false negatives. Recall, also known as Sensitivity or True Positive Rate, may be used. Recall may represent the proportion of actual positives that were correctly identified by the model. It may quantify the ability of the classifier not to miss any positive cases, e.g., its capacity to correctly detect diseases or abnormalities when present. In healthcare, missing even one case could lead to serious consequences, making Recall a metric worthy of consideration.

In some embodiments, a dashboard may be provided that presents the key performance indicators (KPIs) of the model in an accessible manner, enabling quick comprehension of the model's predictions and their associated confidence levels.

In some embodiments, if a positive prediction is received using the generic cancer model, the patient's data may then be fed to more specific cancer type models (e.g. breast cancer model, colon cancer model, etc.) for evaluation.

When dealing with imbalanced datasets where the number of negative instances significantly outweighs the positive ones, there are trade-offs between Recall and other evaluation metrics such as Precision and F1 Score. To address this challenge, the classification threshold may be adjusted can help optimize Recall while maintaining acceptable precision levels. By lowering the threshold, more positive cases may be detected, improving Recall at the cost of increased false positives. Conversely, raising the threshold may decrease false positives but come with fewer true positives, hence reduced Recall. A multi-layered approach may be employed that safeguards the data at every stage of the process.

A secure script may run locally on the healthcare provider's system to de-identify the patient records before transmitting them to the platform. This script may remove all personally identifiable information (PII and PHI) using techniques such as tokenization, hashing, or encryption. Additionally, it may convert the data into a set of features relevant for ML model inference, maintaining data minimality and reducing potential attack vectors.

Before or after the data has been preprocessed and transformed, it may be securely transmitted over encrypted channels to cloud infrastructure where the ML model is deployed. Since only the feature vectors devoid of any personal identifiers may be transferred outside the healthcare organization's computing environment, the risk of unauthorized access to sensitive patient data is significantly mitigated. The cloud infrastructure may comply with stringent industry standards such as HIPAA, GDPR, and CCPA, ensuring full compliance with regulations governing the handling of protected health information. Advanced access control mechanisms may be utilized to manage who can access the data and perform inferences. Access to patient data may be granted based on role-based permissions, two-factor authentication, and strong password policies. These measures may provide an additional layer of security and help prevent unintended disclosure of sensitive information.

The method of the present disclosure may include three main steps: data ingestion, feature engineering, and machine learning inference. Each component may function independently yet collaboratively, allowing for easy integration of new functionality and technologies. The data ingestion step may handle the acquisition, preprocessing, and transformation of raw data from various sources. It may handle both structured and semi-structured data formats, enabling easy integration of new data types and expanded data coverage. The system may support parallel data processing, which may ensure efficient handling of large datasets and improve overall system performance. Feature engineering may be responsible for extracting meaningful insights from the raw data, transforming it into a format suitable for ML algorithms. The system may employ a pluggable architecture, allowing users to define custom feature extraction methods or incorporate third-party libraries. This flexibility may enable rapid experimentation with new approaches and accommodate changing requirements. The machine learning inference step may include performing predictive analysis and generating outcomes based on the extracted features. Containerization technology may be utilized to simplify deployment, scaling, and management of models. This may facilitate adding new models, updating existing ones, or switching between different versions without affecting the overall system stability.

The system may include microservices architecture, which may enable horizontal or vertical scaling. Horizontal scaling may refer to adding more nodes or services to distribute workloads across multiple servers, while vertical scaling may focus on increasing the capacity of individual nodes. Both strategies may enable efficient management of growing demand and deliver timely results. API-driven architecture may facilitate integration with external systems, including Electronic Health Records (EHRs), diagnostic tools, and other clinical decision support platforms. This openness may enable collaboration and streamlines the exchange of valuable information among stakeholders, ultimately improving the accuracy and effectiveness of the disclosed cancer screening solution.

The AI algorithm of the present disclosure may effectively analyze multi-modal medical data including text from case reports, structured tabular data from blood tests, and unstructured image data from various medical imaging techniques such as X-rays or ultrasounds. This may enable a more comprehensive and accurate cancer screening solution.

Multimodal classification may be utilized. Multimodal classification may refer to the process of training ML models on multiple types of input data simultaneously. A patient may be classified as presenting symptoms indicative of cancer based on their medical history, laboratory results, and medical images. By combining information from these diverse sources, overall accuracy of prediction can be improved.

For processing textual data such as medical case reports, Natural Language Processing (NLP) techniques may be utilized. Methods such as tokenization, part-of-speech tagging, named entity recognition, and/or sentiment analysis may be used to extract meaningful features from text data. These techniques may allow for understanding the context, relationships between entities, emotions conveyed within the text, and overall meaning of the report, which may help determine the presence of cancer risk factors.

Structured tabular data may be analyzed. Such data may be from blood tests, for example. Feature extraction methods such as mean, median, standard deviation, correlation matrices, etc., may be used to transform raw data into numerical representations that can be fed into ML models. Additionally, domain knowledge may to be used to select specific features relevant to cancer diagnosis, such as hemoglobin levels, liver enzymes, and/or white blood cell count.

Image preprocessing techniques may include resizing, normalization, filtering, and/or segmentation, while feature extraction techniques may involve identifying distinctive patterns within images, such as edges, texture, color histograms, shape descriptors, or wavelet coefficients. These extracted features may serve as the input to deep neural networks for further analysis. Deep learning architectures, particularly convolutional neural networks (CNNs), may detect subtle differences in medical images that might indicate cancerous growths.

Once features have been extracted from each modality, fusion techniques may be employed to combine them for final prediction. Simple methods such as simple averaging or ensemble methods such as stacking or transfer learning may be used. The selection of fusion techniques may depend on the nature of the input data and the desired tradeoff between complexity and performance.

In the context of cancer screening, high recall can be important because failing to detect even one instance of cancer could lead to serious consequences. However, increasing recall may come at the cost of increased false positives. To balance both, it may be necessary to determine the optimal threshold value for the model. Typically, higher thresholds reduce false positives but increase missed detections, whereas lower thresholds do the opposite. A thorough understanding of the costs associated with both false negatives and false positives may enable informed decisions regarding threshold selection, ensuring that the model prioritizes capturing as many cancer cases as possible without overwhelming healthcare systems with unnecessary follow-ups.

Recall may be used to assess the performance of any binary classification model, including those used in medical applications such as cancer screening. This metric may become especially important when dealing with critical applications where false negatives can lead to severe consequences. In the context of cancer screening, missing a potential case could result in delayed treatment or even loss of life. However, even a recall as low as 0.45 for the positive class might still be considered acceptable in certain scenarios involving cancer screening models. Although this value may seem relatively low, it may ultimately depend on the balance between specificity and sensitivity required in a particular use case. For instance, if minimizing false negatives is prioritized over false positives, accepting a lower specificity (higher false positive rate) in exchange for increased recall could make sense. This approach may prove beneficial in cases where a delay in diagnosis would pose a more significant risk than unnecessary tests. Additionally, considering that advanced technologies like biopsies and MRI scans can be employed to confirm potential cases flagged by the initial screening, the overall diagnostic process can remain comprehensive.

Interpreting the significance of a recall value may depend heavily on the underlying data distribution and base prevalence rates. When evaluating the performance of a cancer screening model, factors such as disease prevalence within the population being screened and the potential impact of false positives and false negatives should be considered. While a low recall implies that some genuine cases remain undetected, understanding the context around these missed detections—such as their severity and the implications for patient outcomes—can help determine whether this trade-off is acceptable.

A method for screening for cancer and/or chronic conditions using AI/ML may include gathering routine medical encounter data, including clinical notes of encounter, routine blood tests, x-ray chest, ultrasound abdomen, mammogram, and other relevant data; preprocessing the collected data by normalizing, cleaning, and structuring it into a format suitable for analysis; applying AI/ML algorithms to analyze the preprocessed data and identify patterns, trends, and relationships between different variables; generating predictive models based on the analyzed data, which can be used to estimate the likelihood of developing cancer or experiencing a chronic condition, including autoimmune diseases; and integrating the generated predictive models into existing electronic health record systems to enable automated screening and early detection of cancer and chronic conditions, including autoimmune diseases. A computer program product stored on a non-transitory computer readable medium may include instructions executable by a processor to perform the method.

An apparatus for screening for cancer and/or chronic conditions using AI/ML may include at least one non-transitory memory; at least one processor; and an application, stored in the at least one non-transitory memory, that when executed by the at least one processor receives routine medical encounter data, including clinical notes of encounter, routine blood tests, x-ray chest, ultrasound abdomen, mammogram, and other relevant data, preprocesses the collected data by normalizing, cleaning, and structuring it into a format suitable for analysis, applies AI/ML algorithms to analyze the preprocessed data and identify patterns, trends, and relationships between different variables, and generates predictive models based on the analyzed data, which can be used to estimate the likelihood of developing cancer or experiencing a chronic condition, including autoimmune diseases.

A method for updating and refining predictive models used for screening for cancer and chronic conditions using AI/ML may include collecting new routine medical encounter data, including clinical notes of encounter, routine blood tests, x-ray chest, ultrasound abdomen, mammogram, and other relevant data; updating the preprocessing steps to include the newly collected data and any changes in preprocessing methods; refining the AI/ML algorithms used to analyze the updated preprocessed data, incorporating new knowledge and insights gained through continuous improvement of the system; and reevaluating the predictive models and making adjustments based on the refined analyses to improve their accuracy and effectiveness at identifying individuals with an increased risk of developing cancer or experiencing a chronic condition, including autoimmune diseases.

The preprocessing may include applying different preprocessing techniques depending on the type of collected data. The preprocessing may include removing instances of disease including cancer-related terms from the collected data. The preprocessing may include feature extraction. The method may further include feeding extracted features into the AI/ML algorithms and performing cross-validation to select a best AI/ML model. The method may further include training the selected AI/ML model by performing one or more optimization techniques including gradient descent tuning, backpropagation tuning, and hyperparameter tuning. The method may further include evaluating the generated predictive models using one or more of the following metrics: Accuracy, Precision, Recall, F1 Score, Area Under ROC Curve, or Confusion Matrix. The method may further include evaluating the generated predictive models based on external data sets.

The method may further include receiving routine medical encounter data of a user, including one or more of clinical notes of encounter, routine blood tests, x-ray chest, ultrasound abdomen, mammogram, or other relevant data for the user; preprocessing the routine medical encounter data of the user by normalizing, cleaning, and structuring it into a format suitable for analysis; applying the generated predictive models to the preprocessed data of the user; and providing an output of applying the generated predictive models to the preprocessed data of the user via a user interface. The output may include one or more of a probability of the cancer or chronic condition, a mapping of that probability to whether the user is likely to have the cancer or chronic condition, or an interpretation relating to the percentage of users with a similar/same probability score having the cancer or chronic condition versus the percentage of users with a similar/same probability and score not having the cancer or chronic condition. The method may further include executing a secure script on the routine medical encounter data of the user prior to the preprocessing to remove any personally identifiable information (PII) or personal health information (PHI). The routine medical encounter data of the user with any PII or PHI removed may be received via one or more encrypted channels.

The predictive models may be integrated into one or more existing electronic health record systems to enable automated screening and early detection of cancer and chronic conditions, including autoimmune diseases. The application may be further configured to receive routine medical encounter data of a user, including one or more of clinical notes of encounter, routine blood tests, x-ray chest, ultrasound abdomen, mammogram, or other relevant data for the user via an application programming interface (API), preprocess the routine medical encounter data of the user by normalizing, cleaning, and structuring it into a format suitable for analysis, apply the generated predictive models to the preprocessed data of the user, and provide via the API an output of applying the generated predictive models to the preprocessed data of the user to a user interface.

In some examples, the AI/ML model may be improved through retraining. For example, based on first training of an AI/ML model, first processing is performed to provide a first processing result based on first processing parameters. Operation of the overall system may be improved through improving the AI/ML model by retraining the AI/ML model based on the first processing result. For example, an evaluation may be made regarding a quality of the first processing result, such as via comparison to control data, real world testing, statistical or other data analysis, or the like. The evaluation may be provided as feedback to retrain the AI/ML model (e.g., second training), such as to increase accuracy of a subsequent processing result of the AI/ML model, decrease power consumption of the AI/ML mode, decrease a hardware processing resource requirement of the AI/ML model, decrease a processing time of the AI/ML model, or the like. Based on the improvement of the AI/ML model, subsequent processing performed by the AI/ML model may be modified. The improved system may perform second processing to provide a second processing result that is improved compared to the first processing. For example, based on the retraining of the AI/ML model, the improved system may provide a different processing result based on the first processing parameters. In this way, based on the second training of the AI/ML model, the system may be configured to perform second processing to provide a second processing result based on the first processing parameters. In some examples, the second processing result may vary from the first processing result based on the improving of the AI/ML model. In other examples, the second processing result may be substantially the same as the first processing result, but may be provided in less time, using less power, using fewer computing resources, or the like based on the improvement of the AI/ML model.

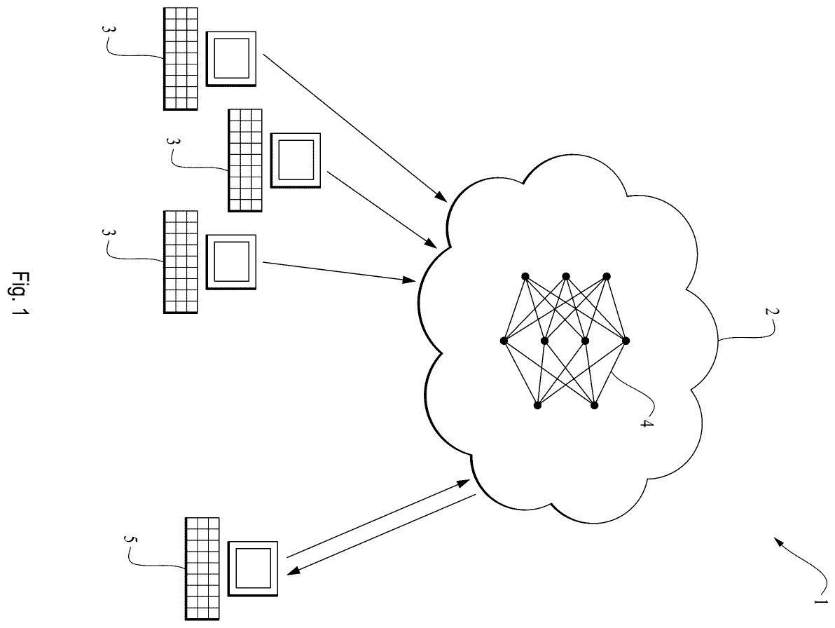

Referring to FIG. 1, a system 1 for training a ML model for screening of a health condition may include one or more processors configured to obtain medical data from a population of patients. For example, the system 1 may include a cloud-based AI system 2 that receives health information from reporting terminals 3. The reporting terminals 3 may be, for example, computers at medical offices or hospitals that collect medical data/health information (e.g., electronic health records (EHR)) about patients. The EHRs may be entered into the reporting terminals 3 manually or automatically. The reporting terminals 3 may be distributed across a country or across the world and/or may also perform functions other than reporting. In some embodiments, the reporting terminals 3 are databases containing electronic medical data/health information. The medical data may be filtered to remove identifying information (e.g., information that might reveal the identity of the patient) before it is sent to the AI system 2. The medical data may be sent via the internet (e.g., in an encrypted manner) to the AI system 2 for training and analysis. Some of the medical data can be used for training the model, and some of the medical information can be used for validating (testing) the model.

The AI system 2 may pre-process the medical data to remove indicia of a health condition (e.g., cancer), label encounters of the medical data according to whether the health condition is present, and train a ML model 4 on the pre-processed, labeled medical data to screen for (e.g., estimate the probability of) the health condition. The ML model 4 may be, for example, a neural network. A diagnostic terminal 5 may communicate with the AI system 2 to screen or diagnose a health condition of a patient using the trained ML model 4. For example, the diagnostic terminal 5 may send (e.g., via the internet) medical data (e.g., EHR) about a patient to the AI system 2. The medical data may be processed to remove identifying information before it is sent. The AI system 2 may use the trained ML model 4 to predict the likelihood that the patient has the health condition. The AI system 2 may then send a report that includes the likelihood that the patient has the health condition via the internet to the diagnostic terminal 5. Although the reporting terminals 3 and the diagnostic terminal 5 have been referred to according to the tasks they perform in this particular example, in some embodiments, there may be computers that perform the functions of both reporting terminals and diagnostic terminals.

In some embodiments, a graphical user interface is provided on the diagnostic terminal 5 which facilitates interaction with the AI system 2. The identifying information may be added back so that the graphical user interface displays the identifying information of the patient along with the screening result. The screening result may be displayed in terms of a diagnosis, an inference of a diagnosis, or a percent likelihood of a diagnosis. In some embodiments, a patient is tested and/or treated based on the screening result. For example, patient data may be fed to the trained ML model 4, and the trained ML model 4 may output a screening result indicating whether cancer is present. If the screening result from the model is positive for cancer, the clinician may prescribe tests for definitive diagnosis, for example, a laboratory technique such as a blood or urine test or a biopsy; an imaging technique such as an X-ray, a CT scan, a MRI, or a PET scan; or an exploratory procedure such as an endoscopic procedure. In some cases, the diagnostic test may be specifically directed at a particular portion of the patient's body, for example, a particular organ; additionally or alternatively, the diagnostic test may be directed at the patient's body more broadly. Additionally, if the diagnosis is confirmed by such tests, then appropriate therapy may be planned. Thus, the patient may (eventually) be treated for cancer in response to (as a result of) receiving the screening result. For example, surgery or chemotherapy may be applied (after additional testing) to the patient in response to the screening result that the patient has cancer.

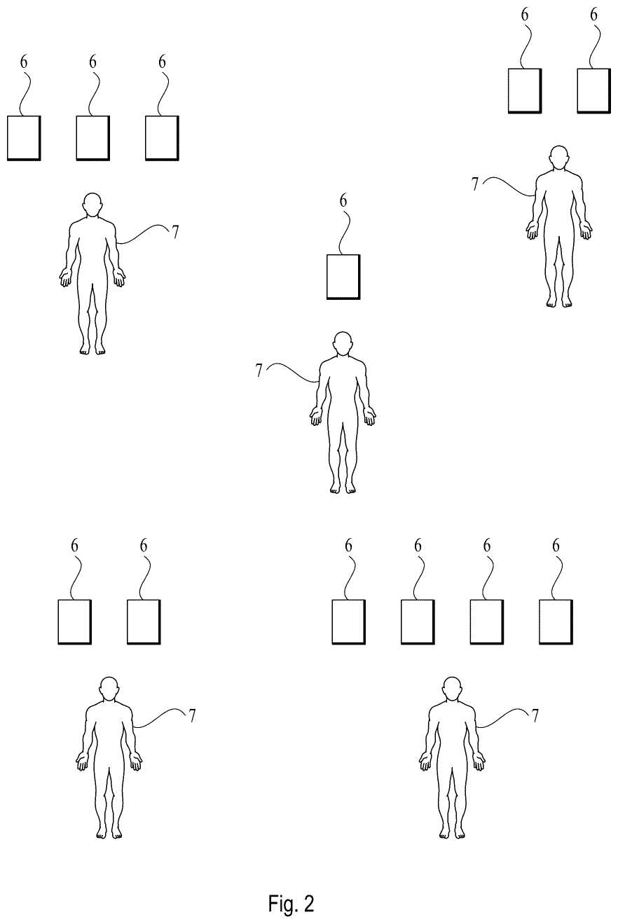

Referring to FIG. 2, the medical data may be organized into encounters 6 which may each be associated with a respective health check of a respective patient 7. An encounter 6 may be all health information taken during a single session or group of associated sessions. For example, an encounter 6 may include health information collected from an initial appointment with a patient 7 and an X-ray that was taken one week later in connection with the appointment. A next encounter 6 associated with a patient 7 may include information from a follow-up appointment three months later and another X-ray taken in connection with the follow-up appointment. Medical data of patients 7 may be separated into encounters 6 based on timing of the information and/or association of the information. For example, if medical data is entered more than one month from the previous entry, it may be considered a new encounter 6. Also, if the health information involves an unrelated topic, but occurs in short succession, it may also be considered a new encounter 6. The rules for separating the health data into encounters 6 may be set according to any suitable approach that allows for the possibility of a change in health status of the patient 7 between encounters 6. As can be seen in FIG. 2, for some patients 7, there is only one encounter 6. For example, after an initial appointment, the patient 7 may be told that no follow-up appointments are necessary. In another example, the patient 7 is told that another follow-up appointment is necessary but that follow-up appointment has not happened yet (thus, there is still only one encounter 6 for that patient 7). For some patients 7, there may be multiple encounters 6. For example, some patients 7 may have two, three, four, or more encounters 6.

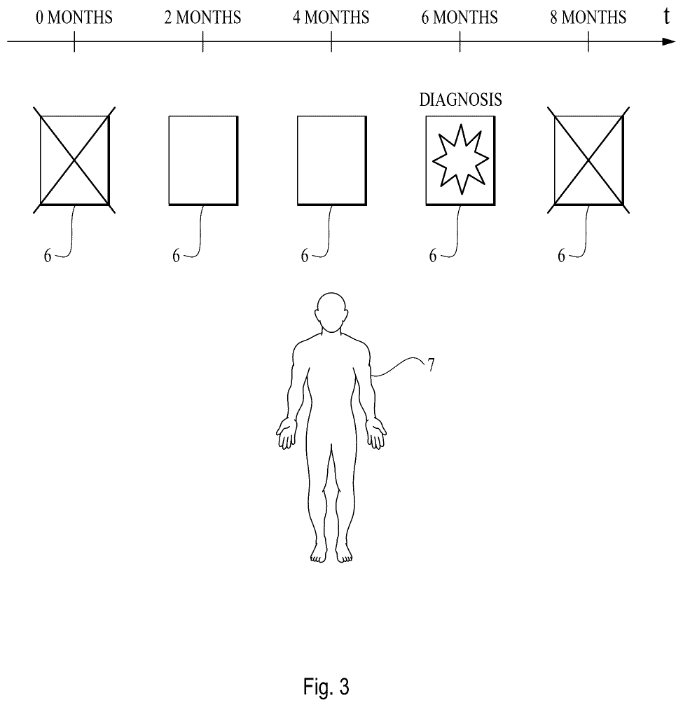

Referring to FIG. 3, the one or more processors may be further configured to, in response to an encounter 6 of the respective patient 7 being labeled as positive for the health condition, label all prior encounters 6 of the respective patient 7 within a time period of the encounter 6 as positive for the health condition, and exclude all prior encounters 6 of the respective patient 7 outside of the time period from the training. In the example of FIG. 3, the patient 7 is diagnosed with the health condition at six months, and the time period is five months. Thus, the encounters 6 at two months and four months may be labeled as positive for the health condition, and the encounter 6 at zero months may be excluded from the training. The one or more processors may be further configured to, in response to an encounter 6 of the respective patient 7 being labeled as positive for the health condition, exclude all subsequent encounters 6 of the respective patient 7 from the training. In the example of FIG. 3, the encounter 6 at eight months occurs after the diagnosis at six months, and thus, the encounter 6 at eight months is excluded from the training. This example is not intended to be limiting. Any suitable time period is within the scope of the present disclosure. For example, the time period could be one, two, three, four, five, six, seven, eight, or more months. Alternative algorithms for labeling or excluding the encounters 6 may be implemented depending on the application.

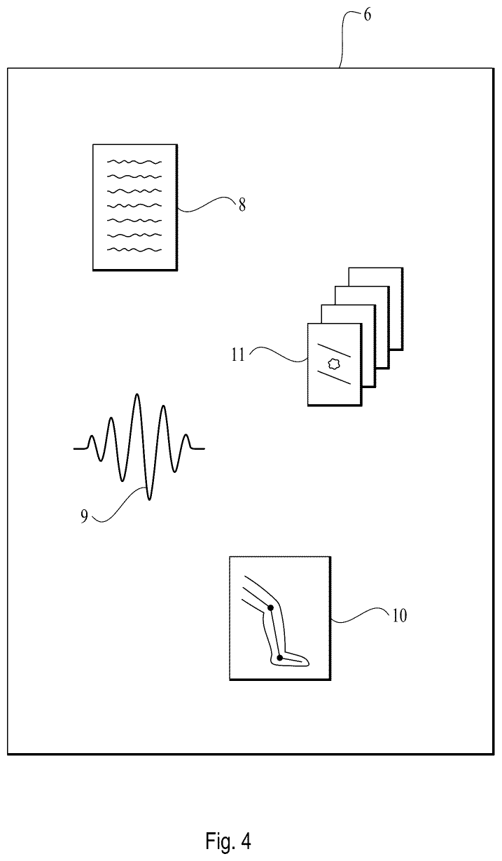

Referring to FIG. 4, each encounter 6 may have a single type of medical data or multiple types of medical data. The medical data may include text information 8 (e.g., summaries of statuses of the patients written by healthcare professionals), audio information 9 (e.g., audio data of the patients), image information 10 (e.g., image data of the patients), and/or video information 11 (e.g., video data of the patients). In some embodiments, each encounter 6 may include text information 8 and at most one image 10. The image 10 may be normalized (e.g., to have a standardized pixel count).

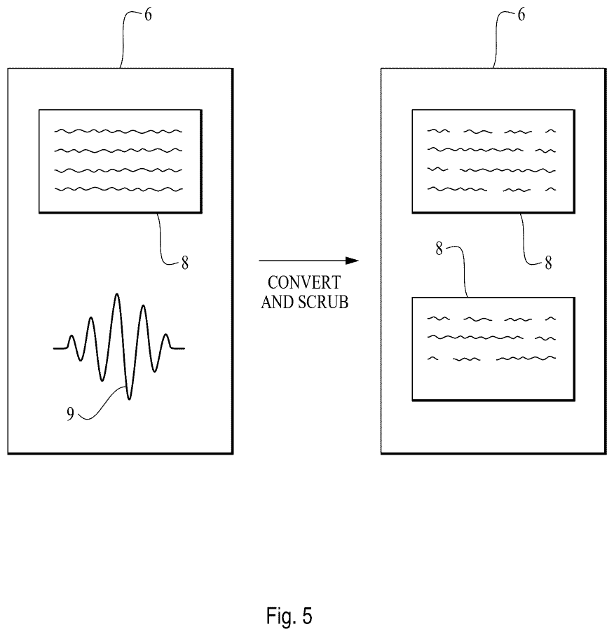

Referring to FIG. 5, the one or more processors may pre-process the medical data to remove indicia of the health condition from the encounters 6. The indicia of the health condition may include diagnoses of the health condition and information that indicates that the health condition is present. The information that indicates that the health condition is present may include a medical procedure necessitated by the health condition and/or a symptom of the medical condition and/or a physical indication that the patient has been treated by the medical procedure. In the example of FIG. 5, the encounter 6 includes text information 8 (e.g., a summary of an appointment written by a physician). The one or more processors may pre-process the medical data by scrubbing the indicia of the health condition from the medical data using a rule-based algorithm. For example, after the process of converting and scrubbing, all mentions of “cancer” and other words that indicate or imply that cancer is present may be removed from the text information 8 but other words in the text information 8 that do not indicate or imply that cancer is present may be kept in the text information 8. In the example of FIG. 5, the encounter 6 also includes audio information 9 (e.g., an audio recording from an appointment with the patient). The audio information 9 may be converted to text information 8 (e.g., using speech recognition software). The text information 8 may also be scrubbed to remove all mentions of “cancer” and other words that indicate or imply that cancer is present. Thus, in this example, an encounter 6 containing text information 8 and audio information 9 may be converted and scrubbed so as to contain only text information 8 that does not include words that indicate or imply that cancer is present. This may be advantageous for more effective training of the ML model.

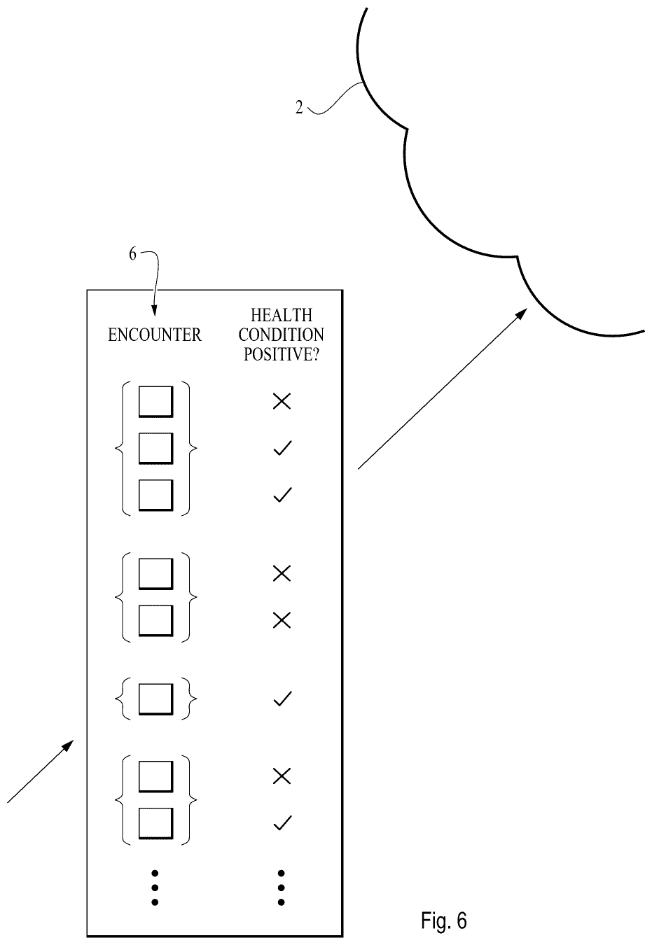

Referring to FIG. 6, the one or more processors may be further configured to label the encounters 6 of the medical data according to whether the health condition is positive or negative, and provide this information to the AI system 2. The encounters 6 may be grouped by patient (as shown by the curly brackets). In the example of FIG. 6, the first three encounters 6 are associated with a first patient, the next two encounters 6 are associated with a second patient, the next encounter 6 is associated with a third patient, and the next two encounters 6 are associated with a fourth patient. The encounters 6 may be individually labeled. That is, some encounters 6 for a given patient may be tagged as positive for the health condition and some encounters 6 from the same patient may be tagged as negative for the health condition. In some embodiments, the encounters 6 are labeled by extracting information regarding whether the health condition exists from the medical data and inferring whether the health condition is present at each of the encounters 6 based on the extracted information. This may be the same data that is later scrubbed out before training the ML model. Alternatively, the system may search the medical data for diagnosis codes and label the encounters 6 based on the diagnosis codes. In other embodiments, the diagnostic information may come pre-labeled. That is, extracting the medical data may simply entail reading the medical data to determine whether the encounter 6 is tagged as the health condition being present. In some embodiments, instead of being organized encounter-by-encounter, the medical data for the encounters 6 for each patient is combined and/or summarized.

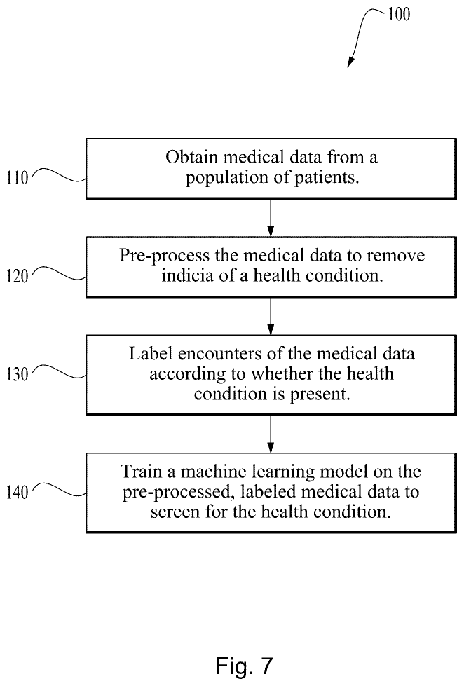

Referring to FIG. 7, a processor-implemented method 100 for training a ML model to screen for a health condition may include the step 110 of obtaining medical data from a population of patients; the step 120 of pre-processing the medical data to remove indicia of a health condition; the step 130 of labeling encounters of the medical data according to whether the health condition is present; and the step 140 of training a ML model on the pre-processed, labeled medical data to screen for/predict/estimate the probability of/detect the health condition.

The medical data may be obtained via an internet reporting system. The medical data may include summaries of statuses of the patients written by healthcare professionals, audio data of the patients, image data of the patients, and/or video data of the patients. The medical data is not limited only to information related to making a diagnosis, but may include other information as well, such as diet and lifestyle information. The medical data may be updated after each visit by the patient. This data may be organized into encounters, and each patient of the population of patients may have one or more encounters. In some embodiments, instead of being organized into encounters, the medical data is organized according to visits. Any disclosure herein related to encounters can also be applied to visits.

The indicia of the health condition may include diagnoses of the health condition and information that indicates that the health condition is preset. The scrubbing of cancer related terms may primarily affect the latter sections of a medical summary/note. This may be because the recommended investigations, findings of some specific investigations such as biopsies, and diagnosis and treatment planning may have most of the terms that are likely to confirm the disease (e.g., cancer). The earlier sections of the medical summary/report may include the medical history, symptoms, examination findings, family medical history, social history, and/or investigation findings from non-specific tests that are done routinely for most patients such as complete blood count. Chest X-rays may be non-specific for cancer and may be minimally affected by the scrubbing for cancer terms. The aim may be to use subtle patterns in these earlier sections to get the ML model to distinguish between presence and absence of cancer.

In some embodiments, if a physician writes in a report that a patient has cancer, the words indicating that the patient has cancer would be removed from the medical data. The information that indicates that the health condition is present may include a medical procedure necessitated by the health condition or a symptom of the medical procedure. For example, if a physician writes in a report that the patient has undergone chemotherapy, the words related to chemotherapy may be removed from the medical data. The pre-processing of the medical data may include scrubbing the indicia of the health condition from the medical data using a rule-based algorithm. The rule-based algorithm may rely on rules to determine which words to selectively remove. The algorithm may use natural language processing to detect instances of the words to be removed. In addition to removing the indicia of the diagnostic information, the rule-based algorithm may remove filler words, articles, or any other words that are not relevant to health. In some embodiments, context is considered when removing a word.

The labeling of the encounters of the medical data may include extracting information regarding whether the health condition exists from the medical data, and inferring whether the health condition is present at each of the encounters based on the extracted information. This may be the same or overlapping information to the information that is removed. Alternatively, the medical data may come pre-labeled according to whether the patient has been diagnosed with the health condition for given encounters. Each of the encounters may be associated with a respective health check of a respective patient of the patient. The encounters may be delineated according to time between healthcare events/appointments and/or main topic of the healthcare events/appointments. The method 100 may further include, in response to a most recent encounter of the respective patient being labeled as positive for the health condition, labeling encounters of the respective patient within a time period of the most recent encounter as positive for the health condition, and excluding all encounters of the respective patient outside of the time period from the training.

The method 100 may further include, in response to an encounter of the respective patient being labeled as positive for the health condition, excluding all subsequent encounters of the respective patient from the training. The method 100 may further include, in response to an encounter of the respective patient being labeled as negative for the health condition, labeling all prior encounters of the respective patient as negative for the health condition.

The cumulative effect of the preprocessing of all of the different types of data may result in great improvements in overall efficiency for the system. The method of processing multi-modal input data described herein may enhance learning outcomes for the ML model due to the rich, complementary data, which may lead to better context understanding.

The system and method of the present disclosure, through the steps of pre-processing the medical data to remove indicia of a health condition and labeling encounters of the medical data according to whether the health condition is present, may result in improved accuracy of health condition screening as compared with conventional screening methods. This improved screening may result in improve patient outcomes for treatment and prevention of medical conditions.

While embodiments have been shown and described, modifications thereof can be made by one skilled in the art without departing from the spirit and teachings of this disclosure. The embodiments described herein are exemplary only and are not intended to be limiting. Many variations and modifications of the embodiments disclosed herein are possible and are within the scope of this disclosure. For example, the various elements or components may be combined or integrated in another system or certain features may be omitted or not implemented. Also, techniques, systems, subsystems, and methods described and illustrated in the various embodiments as discrete or separate may be combined or integrated with other techniques, systems, subsystems, or methods without departing from the scope of this disclosure. Other items shown or discussed as directly coupled or connected or communicating with each other may be indirectly coupled, connected, or communicated with. Method or process steps set forth may be performed in a different order. The use of terms, such as “first,” “second,” “third” or “fourth” to describe various processes or structures is only used as a shorthand reference to such steps/structures and does not necessarily imply that such steps/structures are performed/formed in that ordered sequence (unless such requirement is clearly stated explicitly in the specification).

Disclosure of a singular element should be understood to provide support for a plurality of the element. It is contemplated that elements of the present disclosure may be duplicated in any suitable quantity.

The scope of protection is not limited by the description set out above but is only limited by the claims which follow, that scope including all equivalents of the subject matter of the claims. Each and every claim is incorporated into the specification as embodiments of the present disclosure. Thus, the claims are a further description and are an addition to the embodiments of the present disclosure. Any discussion of a reference herein is not an admission that it is prior art. Any disclosures of all patents, patent applications, and/or publications cited herein are hereby incorporated by reference, to the extent that they provide exemplary, procedural, or other details supplementary to those set forth herein.

As used herein, the term “or” does not require selection of only one element. Thus, the phrase “A or B” is satisfied by either one or both elements from the set {A, B}. A clause that recites “A or B” can be infringed with only one of the listed items, both of the listed items, multiples of the listed items, and one or both of the listed items and another item not listed. The phrase “A, B, or C” is satisfied by any one or any combination of any two or more from the set {A, B, C}. A clause that recites “A, B, or C” can be infringed with only one of the listed items, multiples of the listed items, and one or more of the items from the list and another item not listed.

As used herein, the article “a” means “one or more.” As used herein, the article “an” means “one or more.” As used herein, the article “the” when referring to a singular noun means “the one or more.” Thus, the phrase “an element” means “one or more elements;” and the phrase “the element” means “the one or more elements.”

As used herein, the term “and/or” includes any combination of the elements associated with the “and/or” term. Thus, the phrase “A, B, and/or C” includes any of A alone, B alone, C alone, A and B together, B and C together, A and C together, or A, B, and C together.

What is claimed is:

1. A processor-implemented method for training a machine learning model to screen for a health condition, comprising:
obtaining medical data from a population of patients, wherein the medical data comprises text data, audio data, and image data, and wherein the text data, audio data, and image data are associated with encounters of the patients;
pre-processing the medical data, comprising:
removing indicia of a health condition from the text data;
converting the audio data to text, and removing the indicia of the health condition from the text; and
extracting features from the image data;
labeling the encounters according to whether the health condition is present by extracting information regarding whether the health condition exists from the medical data, and inferring whether the health condition is present at each of the encounters based on the extracted information; and
training a machine learning model on the pre-processed, labeled medical data to screen for the health condition.

2. The method of claim 1, wherein the machine learning model is used to screen a patient for the health condition based on medical data of the patient.

3. The method of claim 1, further comprising updating the machine learning model by training the machine learning model on additional pre-processed, labeled medical data.

4. The method of claim 1, wherein the health condition comprises cancer.

5. A processor-implemented method for training a machine learning model to screen for a health condition, comprising:
obtaining medical data from a population of patients;
pre-processing the medical data to remove indicia of a health condition;
labeling encounters of the medical data according to whether the health condition is present; and
training a machine learning model on the pre-processed, labeled medical data to screen for the health condition.

6. The method of claim 5, wherein the medical data comprises summaries of statuses of the patients, audio data of the patients, image data of the patients, or video data of the patients, wherein the indicia of the health condition comprise diagnoses of the health condition and information that indicates that the health condition is present, and wherein the information that indicates that the health condition is present comprises a medical procedure necessitated by the health condition or a symptom of the medical condition.

7. The method of claim 5, wherein the pre-processing of the medical data comprises scrubbing the indicia of the health condition from the medical data using a rule-based algorithm.

8. The method of claim 5, wherein the labeling of the encounters of the medical data comprises extracting information regarding whether the health condition exists from the medical data, and inferring whether the health condition is present at each of the encounters based on the extracted information.

9. The method of claim 5, wherein each of the encounters is associated with a respective health check of a respective patient of the patients.

10. The method of claim 9, further comprising, in response to a most recent encounter of the respective patient being labeled as positive for the health condition, labeling encounters of the respective patient within a time period of the most recent encounter as positive for the health condition, and excluding all encounters of the respective patient outside of the time period from the training.

11. The method of claim 9, further comprising, in response to an encounter of the respective patient being labeled as positive for the health condition, excluding all subsequent encounters of the respective patient from the training.

12. The method of claim 9, further comprising, in response to an encounter of the respective patient being labeled as negative for the health condition, labeling all prior encounters of the respective patient as negative for the health condition.

13. A system for training a machine learning model to screen for a health condition, comprising:
one or more processors configured to:
obtain medical data from a population of patients;
pre-process the medical data to remove indicia of a health condition;
label encounters of the medical data according to whether the health condition is present; and
train a machine learning model on the pre-processed, labeled medical data to screen for the health condition.

14. The system of claim 13, wherein the medical data comprises summaries of statuses of the patients, audio data of the patients, image data of the patients, or video data of the patients, wherein the indicia of the health condition comprise diagnoses of the health condition and information that indicates that the health condition is present, and wherein the information that indicates that the health condition is present comprises a medical procedure necessitated by the health condition or a symptom of the medical condition.

15. The system of claim 13, wherein the one or more processors are further configured to pre-process the medical data by scrubbing the indicia of the health condition from the medical data using a rule-based algorithm.

16. The system of claim 13, wherein the one or more processors are further configured to label the encounters of the medical data by extracting information regarding whether the health condition exists from the medical data, and inferring whether the health condition is present at each of the encounters based on the extracted information.

17. The system of claim 13, wherein each of the encounters is associated with a respective health check of a respective patient of the patients.

18. The system of claim 17, wherein the one or more processors are further configured to, in response to a most recent encounter of the respective patient being labeled as positive for the health condition, label encounters of the respective patient within a time period of the most recent encounter as positive for the health condition, and exclude all encounters of the respective patient outside of the time period from the training.

19. The system of claim 17, wherein the one or more processors are further configured to, in response to an encounter of the respective patient being labeled as positive for the health condition, exclude all subsequent encounters of the respective patient from the training.

20. The system of claim 17, wherein the one or more processors are further configured to, in response to an encounter of the respective patient being labeled as negative for the health condition, label all prior encounters of the respective patient as negative for the health condition.
